(19) United States
(12) Patent Application Publication
(10) Pub. No.: US 2007/0225566 A1
(43) Pub. Date: Sep. 27, 2007

(54) Observation window cleaning device for endoscope

(75) Inventor: Tetsuya Kawanishi, Saitama-Shi (JP)

Correspondence Address: YOUNG & THOMPSON, 745 SOUTH 23RD STREET, 2ND FLOOR, ARLINGTON, VA, 22202, US

(73) Assignee: FUJINON CORPORATION, SAITAMA-SHI (JP)

(21) Appl. No.: 11/727,031

(22) Filed: Mar. 23, 2007

(30) Foreign Application Priority Data
Mar. 24, 2006 (JP) 2006-081831

(51) Int. Cl. A61B 1/12

(52) U.S. Cl. 600/158; 600/157; 600/159

(57) ABSTRACT

A cleaning device for cleaning an observation window (25) installed to an insertion section (2) of a rigid endoscope (1) is equipped with a nozzle (21) to spray selectively cleaning liquid and a pressurized CO₂ gas against the observation window (25), internal conduits (23, 24) through which the cleaning liquid and the CO₂ gas are supplied to the nozzle (21) and external conduits (90,30; 40) detachably connected to the internal conduits (23, 24), respectively, so as to distribute the cleaning liquid and the CO₂ gas into the internal conduits (23, 24), respectively, from a liquid container (60) and a gas container (89) respectively. The gas supply external conduit (40) has a flow path diameter smaller than the liquid supply external conduit (30).

### BACKGROUND OF THE INVENTION

1. Field of the Invention

The present invention relates to a cleaning device for cleaning an observation window provided at a distal end of an endoscope.

2. Description of Related Art

Laparoscopically assisted surgical operations are less invasive than abdominal surgery due to less invasiveness or less physical infliction on human patients because they enable to perform surgery and medical procedures such as resection of affected parts such as tumors of human body cavity walls and organs, excision of organs, sutures, hemostatis and the like without making an abdominal incision. In such a laparoscopic surgical operation, an endoscope or laparoscope is inserted into an abdominal cavity inflated and expanded with a pneumoperitoneum gas through one of guide tubes made up of tracarls put in incisions to acquire an image of the interior of the abdominal cavity for observation. The endoscope has a rigid section at a distal end of an insertion section which is inserted into the abdominal cavity through the guide tube. This rigid section is equipped with at least an illumination window through which illuminating of the abdominal cavity is made and an observation window through which the illuminated abdominal cavity is observed. For this reason, the observation window has to be kept clean. One of problems which the endoscope encounters in an abdominal cavity is adhesion of dirt such as body liquids to a window glass during endoscopy. In order to keep the observation window clean in an abdominal cavity, the endoscope is typically equipped with a cleaning device for cleaning the observation window as needed while the insertion section remains in the abdominal cavity. The cleaning of the observation window is performed by spraying a cleaning liquid against the window glass to flush away dirt and then blowing off or removing away drops of the cleaning liquid left on the window glass with a pressurized drop removal gas.

Although air can be used for the drop removal gas in the case where the endoscope is used for upper and lower digestive organs, it is a dominant tendency to use not air but a CO₂ gas as well as a pneumoperitoneum gas for blowing off or removing away drops of the cleaning liquid in the case of laparoscopic surgical operations which are applied to enclosed spaces from the viewpoint of patient protection. One of endoscopes which is adapted to blow off or remove away drops of a cleaning liquid left on the window glass with a CO₂ gas is known from, for example, Japanese Patent No. 3359048. This endoscope is provided with an air feed tube through which a CO₂ gas is fed to a gas outlet projecting from a sheath from a gas container filled with the CO₂ gas and an air supply conduit leading to the gas outlet through which the CO₂ gas is introduced into a nozzle provided at a distal end of the sheath. The CO₂ gas is sprayed from the nozzle to blow off drops of the cleaning liquid left on the window glass and dry the window glass.

A CO₂ gas has streaming resistance lower than air in the case of flowing in the air feed tube and supply conduit due to a difference between their viscosity. In consequence, in the case of the endoscope which uses a CO₂ gas in place of air, a rate of CO₂ gas flow is increased as compared with air. This results in oversupply of the CO₂ gas into an abdominal cavity and, accordingly an excessive rise in abdominal pressure which leads to an increase in CO₂ gas absorption.

### SUMMARY OF THE INVENTION

It is therefore an object of the present invention to provide an observation window cleaning device for an endoscope which removes drops of a cleaning liquid from an observation window of the endoscope and dries the observation window without oversupplying a CO₂ gas into an abdominal cavity after cleaning the observation window with the cleaning liquid.

It is another object of the present invention to provide a cleaning device for an endoscope an observation window cleaning device for an endoscope which achieves drying of an observation window of the endoscope with an enhanced effect.

The foregoing object of the present invention is accomplished by an observation window cleaning device for an endoscope having an insertion section which is equipped, at a distal end, with an observation window through which an internal cavity such as an abdominal cavity of a patient is observed and a spray noble through which a cleaning liquid and a CO₂ gas are selectively spouted toward the observation window for cleaning of the observation window with the cleaning liquid or for removal of drops of the cleaning liquid from the observation window with the CO₂ gas after cleaning and further has a liquid supply internal conduit through which the cleaning liquid is supplied to the spray noble and a gas supply internal conduit through which the CO₂ gas is supplied to the spray nozzle. The observation window cleaning device comprises a liquid supply external conduit detachably connected to the liquid supply internal conduit for supplying the cleaning liquid into the liquid supply internal conduit from a liquid container of a pressure pumping type and a gas supply external conduit detachably connected to the gas supply internal conduit for supplying the CO₂ gas into said liquid supply internal conduit from a gas container, wherein the gas supply external conduit has a cross-sectional area smaller than the liquid supply external conduit.

According to the observation window cleaning device for an endoscope of the present invention, it is realized to remove drops of a cleaning liquid from an observation window and dries the observation window without oversupplying a CO₂ gas into an internal cavity such as an abdominal cavity of a patient after cleaning the observation window with the cleaning liquid. Furthermore, wrong connection of the external conduits to the endoscope is reliably prevented. This prevents the operator of the endoscope from being thrown into confusion such as leading to an unfavorable situation from the viewpoint of patient protection.

### BRIEF DESCRIPTION OF THE DRAWINGS

The foregoing and other objects and features of the present invention will be clearly understood from the following detailed description when reading with reference to the accompanying drawings wherein:

FIG. 1 is a schematic view of a rigid endoscope system equipped with an observation window cleaning device according to an embodiment of the present invention;

FIG. 2 is a conceptual view of the a rigid endoscope;

FIG. 3 is a perspective view of a pressure pumping type liquid container with a coupling unit attached thereto; and

FIG. 4 is a conceptual view of the coupling unit attached to the pressure pumping type liquid container.

### DETAILED DESCRIPTION OF THE PREFERRED EMBODIMENTS

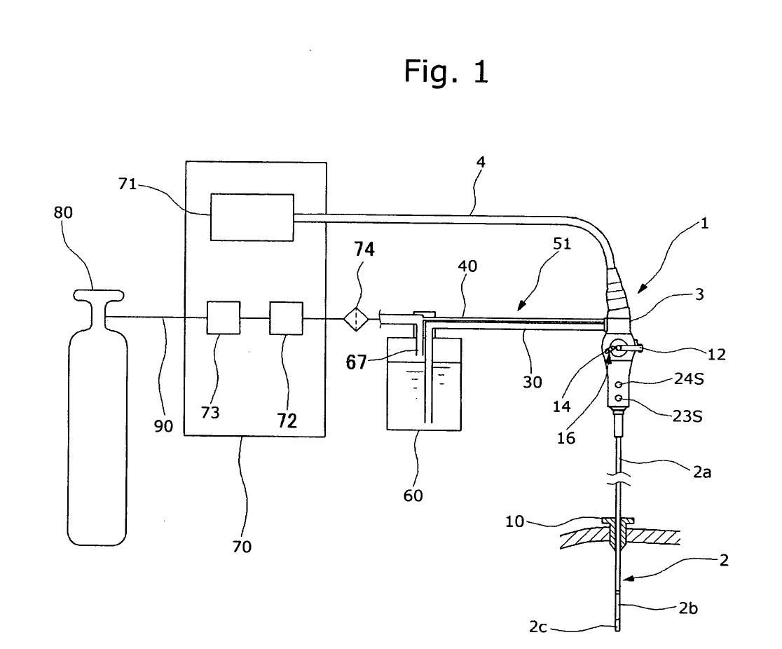

Referring to the accompanying drawings in detail, and in particular, to FIG. 1, a rigid endoscope system equipped with an observation window cleaning device according to an embodiment of the present invention is shown as used for endoscopy and endoscopic procedures. As shown, a rigid endoscope 1 is inserted into a human body cavity, for example an abdominal cavity, of a patient through a guide tube 10 put in an incision in a stomach. The rigid endoscope 1 comprises a rigid insertion section 2 and a manipulation section 3 and is provided with a universal code 4 connected to the manipulation section 3 and a multi-lumen tube 50 detachably connected to the manipulation section 3. The multi-lumen tube 50 comprises a liquid supply external conduit 30 and a gas supply external conduit 40 which are in the form of two lumens (through bores) consolidated integrally. The rigid endoscope 1 is connected to a light source 71 installed within a light source unit 70 through the universal code 4 and a pressure pumping type liquid container 60 containing a cleaning liquid therein through the liquid supply external conduit 30. The liquid container 60 is connected to a gas container 80 containing a CO₂ gas as a drop removal gas therein through a gas distributing conduit 90 such as a high-pressure hose. The gas distributing conduit 90 is connected to a coupling duct 67 extending into the internal space of the liquid container 60. The gas distributing conduit 90 is provided with a safety valve 73, a regulator valve 72 and a filter 74 arranged in order from the gas container 80 and connected to the multi-lumen tube 50 through a three-way coupling unit 69 which will be described later. The safety valve 73 and the regulator valve 72 are desirably installed within the light source unit 70 and may be located at any positions in the gas distributing conduit 90. The rigid endoscope 1 is further connected to a processor unit (not shown) through a universal code.

The insertion section 2 of the rigid endoscope 1 comprises a continuous tubular structure having a rigid portion 2a extending from the manipulation section 3 and making up the major portion of insertion section 2, a flexible portion 2b and a rigid end portion 2c. The flexible portion 2b is bent to head the rigid end portion 2c for a desired direction, namely up and down or right and left. In the case of a flexible endoscope, the major portion 2a is made flexible.

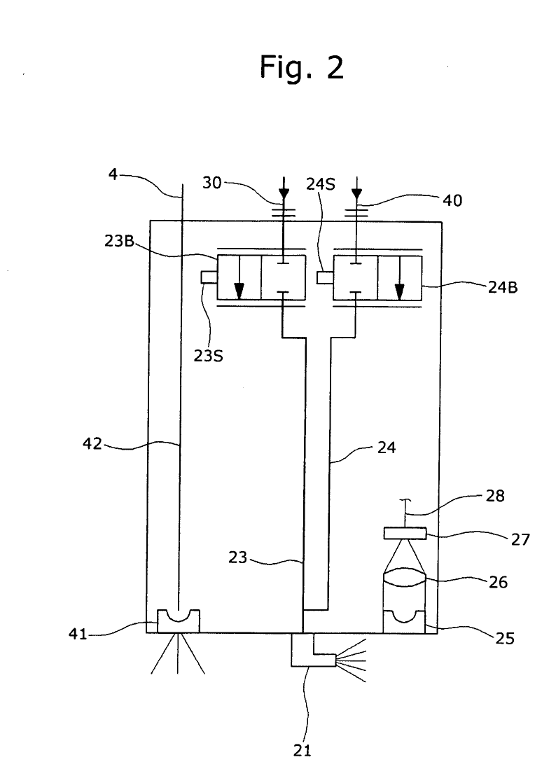

The rigid endoscope 1 is provided with manipulation means 16 installed to the manipulation section 3. The manipulation means 16 includes a manipulation lever 12 which is operated to bend the flexible portion 2b so as thereby to make up and down manipulation or right and left manipulation of the rigid end portion 2c and a lock lever 14 which is operated to lock the flexible portion 2b in a desired bent position. This manipulation means 16 is known in various forms and may take any form known in the art. The rigid endoscope 1 is further provided with a liquid supply button 23S and a gas supply button 24S both installed to the manipulation section 3. The liquid supply button 23S is operated to supply a cleaning liquid to a spray nozzle 21 (see FIG. 2), and the gas supply button 24S is operated to supply a drop removal gas to the spray nozzle 21.

Referring to FIG. 2 schematically showing an internal structure of the rigid endoscope 1, the rigid endoscope 1 is provided with an illumination window 41 including an illumination lens system (not shown), an observation window 25 including a taking lens system 26 and a spray nozzle 21 all of which are installed to the rigid end portion 2c. Light, which is conducted to the illumination window 41 from the light source 71 through a light guide 42 made up of a bundle of optical fibers which is received within the universal code 4 and extends within the rigid endoscope 1, is thrown toward an examination region in an abdominal cavity through the illumination window 41. An optical image of the examination region exposed to the light is gathered though the observation window 25 and focused on an image pickup device 27 such as a solid state image sensor by the taking lens system 26. The image pickup device 27 is known in various forms and may take any form known in the art. The optical image focused on the image pickup device 27 is converted into electric image signals and sent to a signal processing device known as an image processor through an electric line 28. A CO₂ gas and a cleaning liquid are selectively sprayed toward the observation window 25 through the spray nozzle 21. In order to direct a spray of a cleaning liquid and a CO₂ gas toward the observation window 25, the spray nozzle 21 projects from the distal end of the rigid end portion 2c. Specifically, the cleaning liquid in the liquid container 60 is introduced into the spray nozzle 21 through the liquid supply external conduit 30 and a liquid supply internal conduit 23 extending within the insertion section 2 which are connected by way of a liquid supply valve 23B installed within the manipulation section 3. The CO₂ gas in the gas container 80 is introduced into the spray nozzle 21 through the gas distributing conduit 90 and the gas supply external conduit 40 and a gas supply internal conduit 24 extending within the insertion section 2 which are connected by way of a gas supply valve 24B installed within the manipulation section 3. The liquid supply internal conduit 23 and the gas supply internal conduit 24 are united with each other near by the spray nozzle 21. When the liquid supply button 23S, that is installed to the manipulation section 3, is pushed to open the liquid supply valve 23B, the cleaning liquid is sprayed as a jet under a specified pressure through the spray nozzle 21 to clean the observation window 25. On the other hand, when the gas supply button 24S, which is that is installed to the manipulation section 3, is pushed to open the gas supply valve 24B, the CO₂ gas is spouted as a jet under a specified pressure through the spray nozzle 21 to blow off drops of the cleaning liquid left on the observation window 25. When neither the liquid supply button 23S nor the gas supply button 24S is pushed, both supply valves 23B and 24B remain closed, neither the cleaning liquid nor the CO₂ gas is sprayed from the spray nozzle 21.

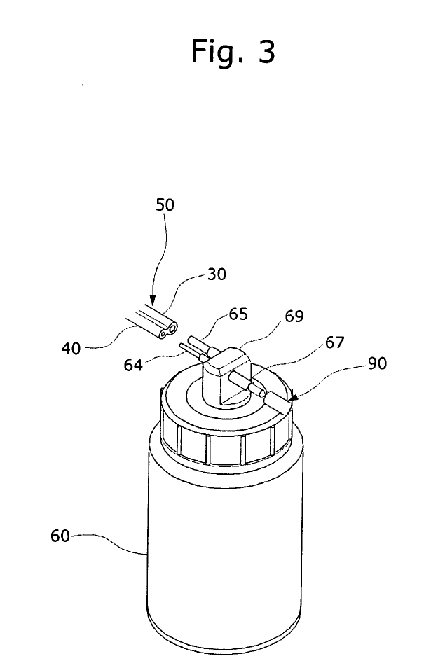

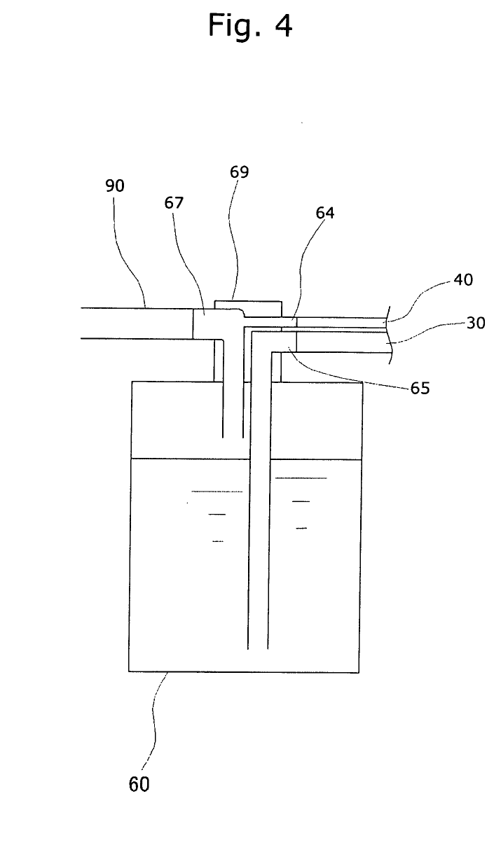

Referring to FIGS. 3 and 4 showing a conduit coupling structure in detail, the three-way coupling unit 69 which is detachably attached to the liquid container 60 has the coupling duct 67, and a coupling duct 64 and a coupling duct 65, besides. These coupling ducts may desirably be formed as integral parts of the three-way coupling unit 69. The coupling duct 67, to which the distributing conduit 90 is connected, branches off into two branch duct portions, one of which forms the coupling duct 64 to which the gas supply external conduit 40 is connected and the other of which forms a gas outlet duct 66 extending into the liquid container 60. The coupling ducts 64 and 65 are arranged side by in conformity with the external conduits 40 and 30 consolidated as the multi-lumen tube 50, respectively, so as to be connected to the gas supply external conduit 40 and the liquid supply external conduit 30, respectively. The coupling duct 65 at one end is submerged in a cleaning liquid such as saline in the liquid container 60. In this instance, the gas supply external conduit 40, and hence the coupling duct 64, has a flow path diameter, namely an inner diameter or a cross-sectional area, smaller than the liquid supply external conduit 30 and its associated coupling duct 65.

When remaining the gas supply valve 24B open by pushing the gas supply button 24S, the CO₂ gas is fed into the gas distributing conduit 90 from in the gas container 80. While flowing through the gas distributing conduit 90, the CO₂ gas is controlled less than a maximum allowable level for safety assurance by the regulator valve 72 and then maintained at a constant pressure level by the safety valve 73. The filter 74 strains out impurities included in the CO₂ gas. The CO₂ gas thus controlled in pressure level and filtered is partly distributed into the liquid container 60 through the coupling duct 67 and partly into the gas supply external conduit 40 through the coupling duct 64. The CO₂ gas introduced into the gas supply external conduit 40 is forced to the spray nozzle 21 through the gas supply internal conduit 24 within the insertion section 2 of the rigid endoscope 1. On the other hand, the CO₂ gas introduced into the liquid container 60 pressurizes the interior of the liquid container 60. When remaining the liquid supply valve 23B open by pushing the liquid supply button 23S, the cleaning liquid in the liquid container 60 is discharged from the pressurized liquid container 60 and forced to the spray nozzle 21 through the liquid supply external conduit 23 within the insertion section 2 of the rigid endoscope 1. By pushing the 23B and 24B alternately, the observation window 25 is washed clean with a jet of the cleaning liquid and hit by a jet of the CO₂ gas, so that drops of the cleaning liquid on the observation window 25 are blown off and the observation window 25 is dried.

As was previously described, the gas supply external conduit 40 has a flow path diameter, namely a cross-sectional area, smaller than the liquid supply external conduit 30 and a CO₂ gas has streaming resistance with respect to the gas supply external conduit 40 lower than air. On the grounds of attributes associated with the CO₂ gas, if the gas supply external conduit 40 is designed without accurate coordination of the cross-sectional area, a problem that is encountered during a surgical operation is that a CO₂ gas is supplied into an abdominal cavity too much to perform the surgical operation with sufficient safety of a patient Conventionally, since a gas supply conduit for use with air for a drop removal gas are designed with the intention to secure a cross-sectional area sufficient enough to pressurize a cleaning liquid in the liquid container, it has not been focus on dimensional coordination between a gas supply conduit and a liquid supply conduit. In general, there are two somewhat conflicting requirements that govern a cross-sectional area of the air supply conduit in the case of using a CO₂ gas which has viscosity lower than air for the drop removal gas. Specifically, the gas supply external conduit 40 requires a cross-sectional area as large as providing a large quantity of CO₂ gas sufficiently enough to apply pressure on the cleaning liquid in the liquid container 60 and as small as possible in order to prevent an abdominal cavity from being filled with a CO₂ gas in excess. According to the observation window cleaning device of the present invention, these conflicting requirements are met by making the gas supply external conduit 40 smaller in cross-sectional area than the liquid supply external conduit 30. It is preferred for these supply external conduit s 30 has a cross-sectional area approximately 1.5 to 2.5 times as large as the gas supply external conduit 40.

In this instance, details of the gas supply external conduit 40 are designed in light of prevention of excessive supply of a CO₂ gas into an abdominal cavity and sufficient pressurization of a cleaning liquid in the liquid container. Besides, the cross-sectional area of the gas supply external conduit 40 is determined in connection with dimensions of the spray nozzle 21. What is a primary factor in liquid drop removal and drying of the observation window 25 is rather sprayed gas pressure or velocity than not sprayed gas quantity at the spray nozzle 21. That is, it is hard to remove drops of a cleaning liquid from the observation window 25 satisfactorily successfully even if a CO₂ gas is sprayed in large quantity at low pressure. In other words, it is essential to spray a CO₂ gas against the observation window 25 at a high velocity or high pressure. Consequently, the gas supply external conduit 40 having a reduced cross-sectional area as small as possible so as thereby to cause a CO₂ gas having relatively low streaming resistance with respect to the gas supply external conduit 40 to flow through the gas supply external conduit 40 maintaining high pressure. In particular, since the pressurization effect of CO₂ gas due to the reduction in cross-sectional area of the gas supply external conduit 40 is enhanced as the gas supply external conduit 40 increases in length. The gas supply external conduit 40, which is generally long in allover length, causes a CO₂ gas to be sprayed as much as sufficient in quantity at sufficiently high pressure through the spray nozzle 21. Therefore, the observation cleaning device performs removal of drops of cleaning liquid from the observation window 25 with an enhanced drop removal effect

Referring to FIG. 4, the gas distributing conduit 90, and hence the coupling duct 67 connected to the gas distributing conduit 90, is configured to have a flow path diameter or cross-sectional area larger than the gas supply external conduit 40 in consideration of the following situation. As described above, the CO₂ gas introduced into the gas distributing conduit 90 is partly distributed into the liquid container 60 through the coupling duct 67 in order to pressurize and pump the cleaning liquid into the liquid supply external conduit 30 through the coupling duct 65. On the other hand, the CO₂ gas introduced into the gas distributing conduit 90 is partly distributed to the spray nozzle 21 through the gas supply external conduit 40 via the coupling duct 64 and then the gas supply internal conduit 24 via the gas supply valve 24B. According to the configuration of the conduits, it is impossible to provide a quantity of CO₂ gas sufficient enough to form a jet of the cleaning liquid at the spray nozzle 21 if using a gas distributing conduit 90 having a small inner diameter or cross-sectional area between the gas container 80 and the liquid container 60, it takes a long time to develop pressure at a required level in the interior of the liquid container 60. For this reason, the gas distributing conduit 90, and hence the coupling duct 67, has a cross-sectional area larger than the gas supply external conduit 40 so as to distribute a large quantity of CO₂ gas sufficiently enough to cause prompt and reliable development of a required level of pressure in the liquid container 60. On this account, the internal pressure of the liquid container 60 rises to a level sufficient enough to form a jet of the cleaning liquid at the spray nozzle 21 in a short time. Consequentially, a jet of cleaning liquid is provided by the spray nozzle 21 in a significantly short response time. This is especially advantageous for the rigid endoscope 1 for use with an electrosurgical knife which is reached to an abdominal cavity through an insertion channel (not shown) provided within the insertion section 2. Because, when using the electrosurgical knife, the observation window 25 easily sustains adhesion of an oil film or the like, it is of important that the observation window 25 is frequently cleaned in order to acquire a clear field of vision quickly on each occasion. In the same breath, despite the gas distributing conduit 90 increased in cross-sectional area, there is no occurrence of a spray of an excessive quantity of CO₂ gas into an abdominal cavity through the spray nozzle 21 because the gas supply external conduit 40 extending to the rigid endoscope 1 is rather thin than the gas distributing conduit 90.

For the meanwhile, as shown in FIG. 3, the coupling ducts 64 and 65 of the three-way coupling unit 69 are arranged side by side in conformity with an arrangement the gas supply external conduit 40 and the liquid supply external conduit 30 consolidated an integrated conduit in the multi-lumen tube 50. This multi-lumen tube 50 has a plurality of, two in this embodiment, raceways adjacent in parallel to one another. The use of the multi-lumen tube 50 realizes uncomplicated arrangement of the conduits. Since the multi-lumen tube 50 has two open ends arranged in conformity to the juxtaposed arrangement of the coupling ducts 64 and 65, the multi-lumen tube 50 stands a chance of ending to wrong connection, or inverse connection, with the coupling ducts 64 and 65 by way of trying connection of them confusing right and left of the multi-lumen tube 50 if the external conduits 30 and 40 have the dimensions. When the multi-lumen tube 50 is inversely connected with the coupling ducts 64 and 65, a consequence adverse to an intended button operation takes place. Specifically, supply channels are created between the liquid container 65 and the spray nozzle 21 through the gas supply external conduit 40, the gas supply valve 24B and the gas supply internal conduit 24 and between the gas container 80 and the spray nozzle 21 through the gas distributing conduit 90, the liquid conduit 30, the liquid supply valve 23B and the liquid supply internal conduit 23, respectively. In consequence, when pushing the liquid supply button 23S is pushed with the intention to clean the observation window 25, the CO₂ gas is jetted out toward the observation window 25. On the other hand, when pushing the gas supply button 24S is pushed with the intention to blow off cleaning liquid drops and dry the observation window 25, the cleaning liquid is jetted out toward the observation window 25. An occurrence of such a situation throws the operator into confusion which is unfavorable situation from the viewpoint of patient protection. On the contrary, the observation window cleaning device of the present invention reliably avoids wrong connection of the multi-lumen tube 50 to the coupling ducts 64 and 65 due to a difference in cross-sectional area between the external conduits 30 and 40 and correspondingly between the coupling ducts 64 and 65.

It is to be understood that although the present invention has been described with regard to preferred embodiments thereof, various other embodiments and variants may occur to those skilled in the art, which are within the scope and spirit of the invention, and such other embodiments and variants are intended to be covered by the following claims.

What is claimed is:

1. An observation window cleaning system for an endoscope having an insertion section which is equipped, at a distal end of said insertion section, with an observation window through which an internal cavity is observed and a spray nozzle through which a cleaning liquid and a CO₂ gas are selectively spouted toward said observation window for cleaning said observation window with said cleaning liquid or removing drops of said cleaning liquid from said observation window with said CO₂ gas and has a liquid supply internal conduit for supplying said cleaning liquid to said spray nozzle and a gas supply internal conduit for supplying said CO₂ gas to said spray nozzle both of which extend within said endoscope, said observation window cleaning device comprising:
a pressure pumping type liquid container for containing said cleaning liquid therein;
a gas container for containing said CO₂ gas herein;
a liquid supply external conduit detachably connected to said liquid supply internal conduit for supplying said cleaning liquid into said liquid supply internal conduit from said pressure pumping type liquid container;
a gas supply external conduit detachably connected to said gas supply internal conduit for supplying said CO₂ gas into said liquid supply internal conduit from a gas container; and
a gas distributing conduit connected to said gas container for distributing said CO₂ gas partly into said gas supply external conduit and partly into said pressure pumping type liquid container; and
a three-way coupling unite equipped with a coupling duct which is detachably connected to said gas distributing conduit and branches off into two gas outlet portions, one of which introduces said CO₂ gas into said pressure pumping type liquid container and the other of which distributes said CO₂ gas into said into said gas supply external conduit, and a coupling duct which is detachably connected to said liquid supply external conduit and extending into said pressure pumping type liquid container and through which said cleaning liquid is introduced into said liquid supply external conduit;
which wherein said gas supply external conduit has a cross-sectional area smaller than said liquid supply external conduit.

2. An observation window cleaning device for an endoscope as defined in claim 1, wherein said gas distributing conduit has a cross-sectional area larger than said gas supply external conduit.

3. An observation window cleaning device for an endoscope as defined in claim 1, wherein said gas supply external conduit and said liquid supply external conduit is formed as a single multi(two)-lumen conduit.

4. An observation window cleaning device for an endoscope as defined in claim 1, wherein said three-way coupling unite is detachably attached to said pressure pumping type liquid container.

5. An observation window cleaning device for an endoscope as defined in claim 4, wherein said gas distributing conduit has a cross-sectional area larger than said gas supply external conduit.

6. An observation window cleaning device for an endoscope as defined in claim 4, wherein said gas supply external conduit and said liquid supply external conduit is formed as a single multi-lumen conduit.
